SEQUENCE LISTING (60 sequences)

SEQ ID NO: 1 - protein, 275 aa, Bacillus amyloliquefaciens
AQSVPYGVSQIKAPALHSQGYTGSNVKVAVIDSGIDSSHPDLKVAGGASMVPSETNPFQDNNSHGTHVAGTVAALNNSIGVLGVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIQSTLPGNKYGAYNGTSMASPHVAGAAALILSKHPNWTNTQVRSSLQNTTTKLGDSFYYGKGLINVQAAAQ

SEQ ID NO: 2 - protein, 275 aa, Bacillus subtilis
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 3 - protein, 275 aa, Bacillus subtilis; identical to SEQ ID NO: 2

SEQ ID NO: 4 - protein, 274 aa, Bacillus licheniformis [sequence omitted]

SEQ ID NO: 5 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 6 - protein, 275 aa, Bacillus stearothermophilus
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPSGSTALKTVVDKAVSSGIVVAAAAGNEGSSGSSSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 7 - protein, 268 aa, Bacillus sp. YaB
QTVPWGINRVQAPIAQSRGFTGTGVRVAVLDTGISNHADLRIRGGASFVPGEPNISDGNGHGTQVAGTIAALNNSIGVLGVAPNVDLYGVKVLGASGSGSISGIAQGLQWAANNGMHIANMSLGSSAGSATMEQAVNQATASGVLVVAASGNSGAGNVGFPARYANAMAVGATDQNNNRATFSQYGAGLDIVAPGVGVQSTVPGNGYASFNGTSMATPHVAGVAALVKQKNPSWSNVQIRNHLKNTATNLGNTTQFGSGLVNAEAATR

SEQ ID NO: 8 - protein, 380 aa, Bacillus alcalophilus
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPNAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 9 - protein, 380 aa, Bacillus clausii
MKKPLGKIVASTALLISVAFSSSIASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 10 - protein, 275 aa, Artificial Sequence
  Description of Artificial Sequence Subtilisin E variant 1
AQSVPNGISQIKAPALHSQGYTGSNVKVAVLDSGIDSTHPDLNVRGGASIVPSETNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 11 - protein, 275 aa, Artificial Sequence
  Description of Artificial Sequence Subtilisin E variant 2
AQSVPYGISQIKATALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPIGSTALKTVVDKAVSSGIVVAAAAGNEGSSGSTSTVGYPAKYPSTIAVGAVNSSNHRASFSSAGSELDVMAPGVSIQSTLPGGTYGAYNGTSMATPHVAGAAALILSKHPTWTNAQVRDRLESTATYLGNSFYYGNGLINVQAAAQ

SEQ ID NO: 12 - protein, 275 aa, Artificial Sequence
  Description of Artificial Sequence Subtilisin E variant 3
AQSVPYGISQIKAPALHSQGYTGSNVKVAVIDSGIDSSHPDLNVRGGASFIPSETNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 13 - protein, 275 aa, Artificial Sequence
  Description of Artificial Sequence Subtilisin E variant 4
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPTGSTALKTAVDKAVSSGIVVVAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVHAAAQ

SEQ ID NO: 14 - protein, 275 aa, Artificial Sequence
  Description of Artificial Sequence Subtilisin E variant 5
AQSVPYGIPQIKAPALHSQGYTGSNVKVAVIDSGIDSSHPDLNVSGGASFVPSETNPYQDGSSHGTHVAGTIAALNNSIGVLGVSPSASLYAVKVLDSTGSGRYSWIINGIEWAISNNMDVINMSLGGPAGSTALKTVVDKAVSSGIVVAAAAGNGGSSGSTSTVGYPAKYPSTIAVGAVNSNNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 15 - protein, 275 aa, Artificial Sequence
  Description of Artificial Sequence Subtilisin E variant 6
AQSVPYGISQIKAPALHSQGYTGSNVKVAVIDSGIDSSHPDLNVRGGASFVPSETNPYQDSSSHGTHVAGTIAALNNPIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 16 - protein, 275 aa, Artificial Sequence
  Description of Artificial Sequence Subtilisin E variant 7
AQSVPYGISQIKAPALHSQGYTGSNVKVAVIDSGIDSSHPDLNVRGGASFVPSDTNPYQDGSSHGTHVAGTIAALNNSFGVLGVSPSASLYAVKVLDSTGSGRYSWIINGIEWAISNNMDVINMSLGGPTGSTALKTVVDKAVSSGIVVAAAAGNGGSSGSTSTVGYPAKYPSTIAVGAVNSSNQRASFSSAGSELDVMAPGVSIQSTLPGGTYGAHNGTSMATPHVAGAAALILSKHPTWTSAQVRDRLESTATYLGNSFYYGKGLINVQAAAQ

SEQ ID NO: 17 - protein, 275 aa, Artificial Sequence
  Description of Artificial Sequence Subtilisin E variant 8
AQSVPYGISQIKAPALHSQGYTGSNVKVAVIDSGIDSSHPDLNVRGGASFVPSETNPYQDGSSHGTHVAGTIAALNNPIGVLGVSPSASIYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 18 - protein, 275 aa, Artificial Sequence
  Description of Artificial Sequence Subtilisin E variant 9
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGKYSWIINGIEWAISNNMDVINMSLGGPAGSTALKTVVDKAVSSGIVVAAAAGNGGSSGSTSTVGYPAKYPSTIAVGAVNSSNQRASFSSAGSELDVMAPGVSIQSTHPGGTYGAYNGTSMATPHVAGAAALILSKHPTWTNAQVRDRLESTATYLGNSFYYGKGLINVQAAAQ

SEQ ID NO: 19 - protein, 275 aa, Artificial Sequence
  Description of Artificial Sequence Subtilisin E variant 10
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGKYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWANAQVRDRLESTATYLGHSFYYGKGLINVQAAAQ

SEQ ID NO: 20 - protein, 275 aa, Artificial Sequence
  Description of Artificial Sequence Subtilisin E variant 11
AQSVPYGISQIKAPALHSQGYTGSDVKVAVIDSGIDSSHPDLNVRGGASFVPSETNPYQDGSSHGTHVAGTIAALNNSIGVLGVSPSASLYAVKVLDSTGSGKYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 21 - protein, 275 aa, Artificial Sequence
  Description of Artificial Sequence Subtilisin E variant 12
AQSVPYGISQIKATGLHSQGYTGSNVKVAVIDSGIDSSHPDLNVRGGASFVPSDTNPYQDGSSHGTHVAGTIAALNNSIGVLGVSPSASLYAVKVLDSTGSGKYSWIINGIEWAISNNMDVINMSLGGPAGSTALKTVVDKAVSSGIVVAAAAGNGGSSGSTSTVGYPAKYPSTIAVGAVNSSNQRASFSSAGSELDVMAPGVSIQSTLPGGTYGAYNGTSMATPHVAGAAALILSKHPTWTNAQVRDRLEGTATYLGNSFYYGKGLINVLAAAQ

SEQ ID NO: 22 - protein, 275 aa, Artificial Sequence
  Description of Artificial Sequence Subtilisin E variant 13
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGKYSWIINGIEWAISNNMDVINMSLGGPTGSKALKTVVDKAVSSGIVVAAAAGNEGSSGSTSTVGYPAKYPSTIAVGAVSSSNQRASFSSAGSELDVMAPGVSIQSTLPGGTYGAYNGTSMATPHVAGAAALILSKHPTWTNAQVRDRLEGTATYLGNSFYYGKGLINVLAAAQ

SEQ ID NO: 23 - protein, 275 aa, Artificial Sequence
  Description of Artificial Sequence Subtilisin E variant 14
AQSVPYGISQIKAPALHSQGYTGSNVKVAVIDSGIDSSHPDLIVRGGASFVPSETNPYQDSSSHGTHVAGTIAALNNPIGVLGVSPSASLYAVKVLDSTGSGKYSWIINGIEWAISNNMDVINMSLGGPAGSTALKTVVDKAVSSGIVVAAAAGNGGSSGSTSTVGYPAKYPSTIAVGAVNSSNQRASFSSAGSELDVMAPGVTIQSTLPGGTYGAYNGTSMATPHVAGAAALILSKHPTWTNAQVRDRLESTATYLGNSFYYGKGLINVQAAAQ

SEQ ID NO: 24 - protein, 275 aa, Artificial Sequence
  Description of Artificial Sequence Subtilisin E variant 15
AQSVPYGISQIKAPALHSQGYTGSNVKVAVIDSGIDSSHPDLSVRGGASFVPSDTNPYQDGSSHGTHVAGTIAALNNSIGVLGVSPSASLYAVKVLDSTGSGRYSWIINGIEWAISNNMDVINMSLGGPTGSTALKTVVDKAVSGGIVVAAAAGNKDSSGSTSTVGYPAKYPSTIAVGAVNSSNQRASFSSAGSELDVVAPGVSIQSTLPGGTYGAHNGTSMATPHVAGAAALILSKHPTWTNAQVRVRLESTATYLGNSFYYGKGLINVQAAAQ

SEQ ID NO: 25 - protein, 275 aa, Artificial Sequence
  Description of Artificial Sequence Subtilisin E variant 16
AQSVPYGISQIKAPALHTQGYTGSNVKVAVIDSGIDSSHPDLSVRGGASFVPSETNPYQDGSSHGTHVAGTIAALNNSIGVLGVSPSASLYAVKVLDSTGSGRYSWIINGIEWAISNNMDVINMSLGGPTGSTALKTAVDKAVSSGIVVVAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVLAAAQ

SEQ ID NO: 26 - protein, 275 aa, Artificial Sequence
  Description of Artificial Sequence Subtilisin E variant 17
AQSVPYGISQIKATVLLSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPAGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 27 - protein, 275 aa, Artificial Sequence; identical to SEQ ID NO: 13
  Description of Artificial Sequence Subtilisin E variant 18

SEQ ID NO: 28 - protein, 275 aa, Artificial Sequence
  Description of Artificial Sequence Subtilisin E variant 19
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNEGSSGSTSTVGYPAKYPSTIAVGAVSSSNQRASFSSAGSELDVMAPGVTIQSTLPGGTYGAHNGTSMAAPHVTGAAALILSKHPTWTNAQVRDRLESTATYLGNSFYYGKGLINVQAAAQ

SEQ ID NO: 29 - protein, 275 aa, Artificial Sequence
  Description of Artificial Sequence Subtilisin E variant 20
AQSAPYGISQIKAPALHSQGYTGSNVKVAVIDSGIDSSHPDLNVRGGASFVPSDTNPYQDSSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSNNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 30 - protein, 275 aa, Artificial Sequence
  Description of Artificial Sequence Subtilisin E variant 21
AQSVPYGIPQIKAPALHSQGYTGSNVKVAVIDSGIDSSHPDLNVRGGASFVPSGTNPYQDGSSHGTHVAGTIAALNNSIGVLGVSPSASLYAVKVLDSTGSGRYSWIINGVEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 31 - protein, 275 aa, Artificial Sequence
  Description of Artificial Sequence Subtilisin E variant 22
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 32 - protein, 275 aa, Artificial Sequence
  Description of Artificial Sequence Subtilisin E variant 23
AQSVPYGISQIKAPALHSQGYTGTNVKVAVIDSGIDSSHPDLNVRGGASFVPSETNPYQDSSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSNNQRASFSSAGSELDVMAPGVSIQSTLPGGTYGAHNGTSMATPHVAGAAALILSKHPTWTNAQVRDRLESSATYLGNSFYYGKGLINVQAAAQ

SEQ ID NO: 33 - protein, 275 aa, Artificial Sequence
  Description of Artificial Sequence Subtilisin E variant 24
AQSVPYGISQIKAPALHSQDYTGSNVKVAVIDSGIDSSHPDLTVRGGASFVPSDTNPYQDGSSHGTHVAGTIAALNNSIGVLGVSPSASLYAVKVLDSTGSGRYSWIINGIEWAISNNMDVINMSLGGPAGSTALKTVVDKAVSSGIVVAAAAGNAGSSGSTSTVGYPAKYPSTIAVGAVNSNNQRASFSSAGSELDVMAPGVSIQSTLPGGTYGAHNGTSMATPHVAGAAALIISKHPTWTNAQVRDRLESTATYLGNSFYYGKGLINVQAAAQ

SEQ ID NO: 34 - protein, 275 aa, Artificial Sequence
  Description of Artificial Sequence Subtilisin E variant 25
AQSAPYGISQIKAPALHSQGYTGSNVKVAVIDSGIDSSHPDLNVRGGASFVPSDTNPYQDSSSHGTHVAGTIAALNNSIGVLGVSPSASLYAVKVLDSTGSGRYSWIINGIEWAISNNMDVINMSLGGPTGSTALKTVVDKAVSGGIVVAAAAGNAGSSGSTSTVGYPAKYPSTIAVGAVNSNNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 35 - protein, 275 aa, Artificial Sequence
  Description of Artificial Sequence Subtilisin E variant 26
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPTGSTALKTAVDKAVSSGIVVVAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRVSLESTATYLGNSFYYGKGLINVQAAAQ

SEQ ID NO: 36 - protein, 275 aa, Artificial Sequence
  Description of Artificial Sequence Subtilisin E variant 27
AQSVPYGISQIKATVLHSQGYTGSNVKVAVLDSGIDSSHPDLSVRGGASFVPSDTNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 37 - protein, 275 aa, Artificial Sequence
  Description of Artificial Sequence Subtilisin E variant 28
AQSVPYGISQIKAPALHSHGYTGSNVKVAVLDSGIDSSHPDLNVRGGASFVPSDTNPYLDGSPHGTHVAGTIAALNNSIGVLGVSPSASLYAVKVLDSTGSGRNSWIINGIEWAISNNMDVINMSLGGPAGSTALKTVVDKAVSSGIVVAAAAGNGGSSGNTSTVGYPAKYPSTIAVGAVNSNNQRASFSSAGSELDVMAPGVSIQSTLPGGTYGAYNGTSMATPHVAGAAALILSKHPTWTHAQVRDRLESTATYLGNSFYYGKGFIHVQAAAQ

SEQ ID NO: 38 - protein, 275 aa, Artificial Sequence
  Description of Artificial Sequence Subtilisin E variant 29
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGNEWAISNYMDVINMSLGGPAGSTALKTVVDKAVSSGIVVAAAAGNAGSSGSTSTVGYPAKYPSTIAVGAVNSSNHRASFSSAGSELDVMAPGVSIQSTLPGGTYGAYNGTSMATPHVAGAAALNPSKHPTWTNALVRDRLESTATYLGHSFYYGKGLINVQAAAQ

SEQ ID NO: 39 - protein, 275 aa, Artificial Sequence
  Description of Artificial Sequence Subtilisin E variant 30
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGNEWAISNYMDVINMSLGGPAGSTALKTVVDKAVSSGIVVAAAAGNAGSSGSTSTVGYPAKYPSTIAVGAVNSSNHRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 40 - protein, 275 aa, Artificial Sequence
  Description of Artificial Sequence Subtilisin E variant 31
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGQYSWIINGIEWAISNNMDVINMSLGGPAGSTALKTVVDKAVSSGIVIAAAAGNAGSSGSTSTVGYPAKYPSTIAVGAVNSNNQRASFSSAGSELGVMAPGVSIHSTLPGGTYGAYIGTSMATPHVAGAAALILSKHPTWTNAQVRDRLESTATYLGNSFYYGKGLIIVQAAAQ

SEQ ID NO: 41 - protein, 275 aa, Artificial Sequence
  Description of Artificial Sequence Subtilisin E variant 32
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALNTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLIDVQAAAQ

SEQ ID NO: 42 - protein, 275 aa, Artificial Sequence
  Description of Artificial Sequence Subtilisin E variant 33
AQSVPNGISQIKATVLHSQGYTGSNVKVAVLDSGIDSSHPDLSVRGGASFVPSDTNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 43 - protein, 275 aa, Artificial Sequence
  Description of Artificial Sequence Subtilisin E variant 34
AQSVPYGISQIKATVLHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGFPAKYPSTIAVGAVNSSNQRASFSSAGSELDVMAPGVSIQSTLPGGTYGAHTGTSMATPHVAGAAALILSKHPTWTNAQVRDRLESTATYLGNSFYYGKGLINVQAAAQ

SEQ ID NO: 44 - protein, 275 aa, Artificial Sequence
  Description of Artificial Sequence Subtilisin E variant 35
AQSVPYGISQIKATVLHSQGYTGSNVKVAVLDSGIDSSHPDLNVRGGASFVPSDTNPYQDGSSHGTHVAGTIAALNNSIGVLGVSPSASLYAVKVLDSTGSGRYSWIINGIEWAISNYMDVINMSLGGPTGSTALKTVVDKAVSSGIVVAAAAGNAGSSGSTSTVGYPAKYPSTIAVGAVNSSNQRASFSGAGSELDVMAPGVSIQSTLPGGTYGAYNGTSMATPHVAGAAALILSKHPTWTTAQVRDRLESTATYLGNSFYCGKGLIHVQAAAQ

SEQ ID NO: 45 - protein, 275 aa, Artificial Sequence
  Description of Artificial Sequence Subtilisin E variant 36
AQSVPYSIAQIKAPALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSTVGYPAKYPSTIAVGAVNSSNQRASFSSAGSELDVMAPGVSIQSTLPGGTYGAHNGTSMAAPHVAGAAALILSKHPTWTNAQVRDRLESTATYLGNSFYNGKGLINVQPAAQ

SEQ ID NO: 46 - protein, 275 aa, Artificial Sequence
  Description of Artificial Sequence Subtilisin E variant 37
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALNTVVDKAVSSGIVVAAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 47 - protein, 275 aa, Artificial Sequence
  Description of Artificial Sequence Subtilisin E variant 38
AQSVPYGIPQIKAPALHSQGYTGSNVKVAVIDSGIDSSHPDLNVRGGASFVPSETNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSSVGYPAKYPSTIAVGAVNSNNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 48 - protein, 275 aa, Artificial Sequence
  Description of Artificial Sequence Subtilisin E variant 39
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSTVGYPAKYPSTIAVGAVNSSYQRASFSSAGSELDVMAPGVSILSTLPGGTYGAHNGTSMATPHVAGAAALILSKHPTWTNAQVRDRLESTATYLGNSFYYGNGLINVQAAAQ

SEQ ID NO: 49 - protein, 275 aa, Artificial Sequence
  Description of Artificial Sequence Subtilisin E variant 40
AQSVLHGISQIKAPALHSQGYTGSNVKVAVIDSGIDSSHPDLNVRGGAGFVPSETNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVQAAAQ

SEQ ID NO: 50 - protein, 275 aa, Artificial Sequence
  Description of Artificial Sequence Subtilisin E variant 41
AQSVPYGISQIKAPALHSQGYTGSNVKVAVIDSGIDSSHPDLNVRGGASFVPSGTNPYQDGSSHGTHVAGTIAALNNSIGVLGVSPSASLYAVKVLDSTGSGRYSWIINGVEWAISNNMDVINMSLGGPTGSTALKTAVDKAVSSGIVVVAAAGNAGSSGSTSTVGYPAKYPSTIAVGAVNSSNQRASFSSAGSELDVMAPGVSIQSTLPGGTYGAHNGTSMATPHVAGAAALIISKHPTWTNAQVRDRLESTATYLGNSFYYGKGLINVQAAAQ

SEQ ID NO: 51 - protein, 275 aa, Artificial Sequence
  Description of Artificial Sequence Subtilisin E variant 42
AQSVPYGISQIKAPALHSQGYTGSNVKVAVIDSGIDSSHPDLNVRGGASFVPSETNPYQDGSSHGTHVAGTIAALNNSIGVLGVSPSAPLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVHAAAQ

SEQ ID NO: 52 - protein, 275 aa, Artificial Sequence
  Description of Artificial Sequence Subtilisin E variant 43
AQSVPYGISQIKAPALHSQGYTGSNVKVAVLDSGIDSSHPDLSVRGGASFVPSETSPYQDGSSHGTHVAGTIAALNNSIGVLGVSPSASLYAVKVLDSTGSGRYSWIINGIEWAISNNMDVINMSLGGPTGSTALKTAVDKAVSSGIVVVAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVHAAAQ

SEQ ID NO: 53 - protein, 275 aa, Artificial Sequence
  Description of Artificial Sequence Subtilisin E variant 44
AQSVPYGISQIKATVLHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSTALKTVVDKAVSSGIVVAAAAGNAGSSGSTSTVGYPAKYPSTIAVGAVNSSNQRASFSSAGSELDVMAPGVSIQSTLPGGTYGAHNGTSMATPHVAGAAALILSKHPTWTNAQVRDRLESTATYLGNSFYYGKGLINVHAAAQ

SEQ ID NO: 54 - protein, 275 aa, Artificial Sequence
  Description of Artificial Sequence Subtilisin E variant 45
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNAGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 55 - protein, 275 aa, Artificial Sequence
  Description of Artificial Sequence Subtilisin E variant 46
AQSVPYGISQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGIEWAISNNMDVINMSLGGPTGSKALKTVVDKAVSSGIVVAAAAGNEGSSGSTSTVGYPAKYPSTIAVGAVNSSNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 56 - protein, 275 aa, Artificial Sequence
  Description of Artificial Sequence Subtilisin E variant 47
AQSAPYGISQIKAPALHSQGYTGSNVKVAVIDSGIDSSHPDLNVRGGASFVPSDTNPYQDSSSHGTHVAGTIAALNNSIGVLGVSPSASLYAVKVLDSTGSGRYSWIINGIEWAISNNMDVINMSLGGPTGSTALKTVVDKAVSSGIVVAAAAGNEGSSGSTSTVGYPAKYPSTIAVGAVNSNNQRASFSSAGSELDVMAPGVSIQSTLPGGTYGAYNGTSMATPHVAGAAALILSKHPTWTNAQVRDRLESTATYLGNSFYYGKGLINVQAAAQ

SEQ ID NO: 57 - protein, 275 aa, Artificial Sequence
  Description of Artificial Sequence Subtilisin E variant 48
AQSVPYGIPQIKAPALHSQGYTGSNVKVAVIDSGIDSSHPDLNVRGGASFVPSDTNPYQDGSSHGTHVAGTIAALNNSIGVLGVSPSASLYAVKVLDSTGSGRYSWIINGVEWAISNNMDVINMSLGGPTGSTALKTVVDKAVSSGIVVAAAAGNEGSSGSTSTVGYPAKYPSTIAVGAVNSNNQRASFSSAGSELDVMAPGVSIQSTLPGGTYGAYNGTSMATPHVAGAAALILSKHPTWTNAQVRVSLESTATYLGNSFYYGKGLINVQAAAQ

SEQ ID NO: 58 - protein, 275 aa, Artificial Sequence; identical to SEQ ID NO: 24
  Description of Artificial Sequence Subtilisin E variant 49

SEQ ID NO: 59 - protein, 275 aa, Artificial Sequence
  Description of Artificial Sequence Subtilisin E variant 50
AQSVPYGISQIKAPALHSQGYTGSNVKVAVIDSGIDSSHPDLNVRGGASFVPSETNPYQDGSSHGTHVAGTIAALNNSIGVLGVSPSASLYAVKVLDSTGSGQYSWIINGIEWAISNNMDVINMSLGGPTGSTALKTVVDKAVSGGIVVAAAAGNKGSSGSTSTVGYPAKYPSTIAVGAVNSSNQRASFSSAGSELDVMAPGVSIQSTLPGGTYGAYNGTSMATPHVAGAAALILSKHPTWTNAQVHDRLESTATYLGNSFYYGKGLINVQAAAQ

SEQ ID NO: 60 - DNA, 1146 bp, Bacillus subtilis
gtgagaagcaaaaaattgtggatcagcttgttgtttgcgttaacgttaatctttacgatggcgttcagcaacatgtctgtgcaggctgccggaaaaagcagtacagaaaagaaatacattgtcggatttaaacagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgttagcccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtcgttgacaaagccgtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgcaggttctgagcttgatgtgatggctcctggcgtgtccatccaaagcacacttcctggaggcacttacggcgcttataacggaacgtccatggcgactcctcacgttgccggagcagcagcgttaattctttctaagcacccgacttggacaaacgcgcaagtccgtgatcgtttagaaagcactgcaacatatcttggaaactctttctactatggaaaagggttaatcaacgtacaagcagctgcacaataa

(19) United States
(12) Patent Application Publication
(10) Pub. No.: US 2003/0157645 A1
(43) Pub. Date: Aug. 21, 2003

(54) Subtilisin variants with improved characteristics

(75) Inventors: Ulrich Kettling, Koeln (DE); Andre Koltermann, Koeln (DE); Oliver Kensch, Koeln (DE); Rene Kuhlemann, Koeln (DE); Ulrich Haupts, Koeln (DE); Markus Rarbach, Koeln (DE); Konrad Odendahl, Koeln (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: DIREVO Bio Tech AG.

(21) Appl. No.: 10/324,152

(22) Filed: Dec. 19, 2002

Related U.S. Application Data
(60) Provisional application No. 60/343,056, filed on Dec. 21, 2001.

(51) Int. Cl. C11D 3/386; C07H 21/04; C12N 9/56; C12N 1/21; C12P 21/02; C12N 15/74

(52) U.S. Cl. 435/69.1; 510/320; 510/226; 435/222; 435/252.3; 435/320.1; 536/23.2

(57) ABSTRACT

The present invention provides subtilisin variants with improved characteristics, including improved substrate affinity, catalytic activity, catalytic efficiency and stability under washing conditions as well as overall wash performance. The subtilisin variants are therefore useful additives in cleaning compositions.

### FIELD OF THE INVENTION

The present invention provides subtilisin variants with improved characteristics, including improved substrate affinity, catalytic activity, catalytic efficiency and stability under washing conditions as well as overall wash performance. The subtilisin variants are therefore useful additives in cleaning compositions.

### BACKGROUND OF THE INVENTION

Proteases are enzymes that catalyze the hydrolysis of peptide bonds in a protein's backbone. Protease with low substrate specificity are required in order to degrade various protein soils to smaller fragments and, thereby, to support a detergent's action of removing stains like egg, grass, grain etc. from textiles and other materials. Subtilisins are used for this purpose for more than 30 years with large success, mainly as additives in cleaning compositions, e.g. laundry detergents. Consequently, detergent proteases account today for approximately 30% of total worldwide enzyme production. A large number of different subtilisins and subtilisin-like enzymes have been isolated and cloned from several microorganisms including mainly bacillus species (e.g. Bacillus subtilis, Bacillus lentus, Bacillus licheniformis, Bacillus amyloliquefaciens), but also from other organisms (e.g. Tritirachium album, Thermoactinomyces vulgaris). However, only a few subtilisins have been used commercially as additives for cleaning compositions, mainly due to the fact that the technical conditions under which the enzyme is used are far away from natural conditions. For application in cleaning compositions subtilisins need to be stable and active under the harsh conditions of the automatic washing procedure (medium to high temperature, alkaline pH, oxidizing conditions, surfactants, etc.). Consequently, a large number of studies have been done on the design of subtilisins to fulfill these requirements.

Properties of a protease can be improved by introducing mutations into the amino acid sequence of a precursor, subtilisin at defined positions. Such efforts have lead to better subtilisins as disclosed in several patent applications. For example, U.S. Pat. No. 5,346,823 describes subtilisin derivatives which are modified by substitution of methionine, trypthophane, lysine or cysteine with alanine or serine resulting in improved stability under highly oxidizing conditions.

Analogously, a number of subtilisin variants have been obtained that are better suited for technical conditions such as highly alkaline pH, high temperature, high concentrations of detergents, etc. According to U.S. Pat. No. 5,246,849 thermally stable serine proteases are created by introducing cysteine at position 206. Subtilisins with enhanced thermal stability were obtained by modifications at the calcium-binding sites at positions 131 and 172 (see U.S. Pat. No. 5,260,207). Subtilisin variants showing alterations in characteristics as thermal and alkaline stability were described in U.S. Pat. No. 5,972,682. Besides of stability, the utility of an enzyme is further characterized by its catalytical behavior in its proposed technical field. Related parameters such as proteolytic activity, wash performance, were also already subject of several patents. For example, single amino acid substitutions at position 225 and at positions 224 as disclosed in U.S. Pat. No. 5,155,033 and in U.S. Pat. No. 5,182,204, respectively, resulted in at least 1.5 fold enhanced catalytic activity of the modified subtilisin compared to its precursor subtilisin.

Multi-mutations also can result in increased proteolytic activity as disclosed in EP-A-0451244 describing substitutions at position 123 and 274. U.S. Pat. No. 6,287,841 discloses subtilisin mutants derived from PB92 subtilisin or Subtilisin 309 by substitutions at combinations of positions 60, 87, 97, 99, 102, 116, 117, 126, 127, 128, 130, 133, 134, 154, 158, 159, 160, 164, 169, 175, 180, 182, 193, 197, 198, 203, 211 and 216 corresponding to these precursor subtilisins, having improved wash performance or/and improved stability.

As another example, the subtilisins described in WO 91/00345 and EP-A-0405901, with change of the net electrostatic charge in favour to an isoelectric point lower or higher than that of the parent subtilisins, resulting in subtilisins with improved adsorption to the substrate, i.e. protein soils on textile surfaces or other material to be cleaned, encompass among other mutations a modification done at position 54 with the glutamic acid being substituted by Thr, Tyr or Gly. WO 92/11348, as well as U.S. Pat. Nos. 5,631,217 and 5,482,849 disclose the substitution at position 54 by aspartic acid.

U.S. Pat. No. 5,316,935 discloses modifications selected from D60N, D97G, Q103R, G131D, S182G, S188P, D248A, D248L, D248N or T255, and optionally E156G and/or N218S when the subtilisin already contains the substitution N181S resulting in modified subtilisins as catalysts with improved stability and/or catalytic activity in organic solvents.

U.S. Pat. No. 4,760,025 describes subtilisin variants derived from subtilisin naturally produced by Bacillus amyloliquefaciens that are substituted at positions 32, 155, 104, 222, 166, 64, 33, 169, 189, 217, 157 by any different amino acid. U.S. Pat. No. 5,700,676 also pertains to improved subtilisin modified at selected positions by substitution of the amino acid with a different naturally occurring amino acid.

According to U.S. Pat. No. 6,197,567, mutant subtilisins distinguished from the parent subtilisins by different amino acids at one or more selected positions with the different amino acid being more positive or negative than the amino acid in the corresponding position in the parent subtilisin resulting in enzyme mutants exhibiting improved wash performance.

WO 95/07991 describes subtilisin mutants with improved properties as decreased adsorption to and increased hydrolysis of an insoluble substrate, wherein the modifications are defined substitutions in the range of positions 199 to 200, either at least at one position or at two positions. For example, in case of a double substitution there are a number of amino acids proposed substituting the tyrosine at position 217 with the preferred amino acid being leucine. EP-A-0380362, which pertains to the use of subtilisin mutants in chemical reaction in a non-native environment, discloses lysine or leucine as appropriate substituating amino acids at position 217, whereas WO 96/09396 describes a calcium-free thermostable subtilisin mutant comprising the modification Y217K, inter alia.

WO 01/07578 describes subtilisin-like protease variants having decreased immunogenicity evolved by the combination of defined substitutions at one or more positions in one or more epitope regions (ranging from 108 to 126 AA; 221-246 AA), at two or more positions in one or more epitope region (113-125 AA; 221-246 AA), at one or more positions in two or more of the epitope regions (108-125 AA; 221-246 AA; 70-84 AA) or at two or more positions in one or more of the epitope regions (ranging from 103-126 AA, 220-246AA, 70-84 AA).

WO 95/30010 concerns variants with decreased adsorption to and increased hydrolysis of an insoluble substrate and discloses defined amino acid substitutions in six loop regions of BPN′ subtilisin, with the first loop region ranging from AAs 59 to 66, the second loop from AAs 95 to 107, the third loop from AAs 126 to 133, the fourth loop from AAs 154 to 167, the fifth loop from AAs 187 to 191, and the sixth loop from AAs 199 to 220 with the BPN′ variants having modified amino acid sequences comprising one or more substitutions in one or more of the loop regions (first to fifth) or modifications as described within the sixth loop region in combination with substitution(s) in one of the aforementioned loop regions.

WO 95/10615 describes subtilisin variants with altered properties compared to those of the precursor subtilisins having a substitution at position 76 and further substitutions at selected positions (99, 101, 103, 104, 107, 123, 27, 105, 109, 126, 128, 135, 156, 166, 195, 197, 204, 206, 210, 217, 218, 222, 260, 265, 274) with the main substitution being N76D.

WO 99/20770 pertains to subtilisin variants with a substitution at position 103 in combination with one or more substitutions at amino acid residues in the range from 1 to 275, with the main substitution being defined as S103A.

The variants according to U.S. Pat. No. 6,312,936 comprise substitutions at position 103 and 236 and/or 245 and further substitutions within the range from AAs 1 to 275 with the main substitutions being S103A, Q236H, Q246L.

Although a lot of progress has been achieved in the field of subtilisin modifications, there is continuing interest in new protease variants with further enhanced characteristics. One major point of common interest is the still enormous amount of protease—be it engineered or not—needed for the technical application in the cleaning process. Taking laundry detergents as an example, the concentration of protease needed for a sufficient removal of protein stains from textiles is between 1 and 5 mg per liter washing liquid. This leads to a worldwide annual consumption of more than 500.000 kg enzyme per year. Furthermore, the high protease concentration corresponds to an increased chance of exposure to the potentially immunogenic protein and, thereby, to an increased probability of allergenic effects.

Therefore, the underlying problem of the present invention is to provide subtilisin variants with improved characteristics, especially subtilisin variants that can be applied technically as additives in cleaning compositions at significantly lower concentrations as compared to known subtilisin variants. Further improved characteristics may include, but are not limited to, higher catalytic rates under application conditions, higher substrate affinity under application conditions, faster removal of protein stains from the particular surface under application conditions, improved stain removing ability at low laundering temperature, better wash performance, improved stability upon storage, improved stability during use, and improved resistance to self-degradation under application conditions. The present invention provides subtilisin variants that are improved for at least one of these characteristics. These subtilisins are useful additives in cleaning compositions such as laundry detergents and other technical or industrial applications.

### SUMMARY OF THE INVENTION

The Present Invention Provides

(1) a subtilisin variant having improved characteristics under technical conditions such as a laundry detergent solution, in particular a modified subtilisin having at least one mutation in an amino acid sequence of a precursor subtilisin at a position numbered according to the amino acid sequence of the mature subtilisin BPN′ shown in SEQ ID NO:1, wherein said at least one mutation is selected from (a) E54D or E54G, (b) Q103R or Q103K, (c) T133K, (d) E156K or E156A, and (e) Y217H, or mutations at the respective positions which are homologous to said mutations (a) to (e), provided that in case of mutations E54D, Q103R, E156K or E156A the modified subtilisin has at least one more of said mutations (a) to (e);

(2) a modified subtilisin as defined in (1) above which has at least two of the following group of amino acid substitutions: E54D, Q103R, T133K, E156A, Y217H;

(3) a modified subtilisin as defined in (1) or (2) above which is selected from variants 1 to 50 defined hereinbelow;

(4) a DNA coding for the modified subtilisin as defined in (1) to (3) above;

(5) a vector comprising the DNA as defined in (4) above;

(6) a microorganism which comprises the DNA as defined in (4) above and/or the vector as defined in (5) above;

(7) a method for producing the modified subtilisin as defined in (1) to (3) above which comprises culturing a microorganism as defined in (6) above;

(8) a detergent composition comprising a modified subtilisin as defined in (1) to (3) above; and

(9) a method for degrading proteinaceous material which comprises treating the proteinaceous material with a modified subtilisin as defined in (1) to (3) above.

### SHORT DESCRIPTION OF THE FIGURES

FIG. 1 shows a plasmid map of the shuttle vector pBVP43-Sub.

FIG. 2 shows an alignment of several subtilisins of the genus Bacillus, wherein Sub DY is subtilisin DY (Bacillus subtilis; SEQ ID NO:3), Sub BLI is subtilisin Carlsberg (Bacillus licheniformis; SEQ ID NO:4), Sub E is subtilisin E (Bacillus subtilis; SEQ ID NO:2), Sub BPN is subtilisin BPN′ (Bacillus amyloliquefaciens; SEQ ID NO:1) and Sub Sav is subtilisin Savinase (Bacillus lentus; SEQ ID NO:5).

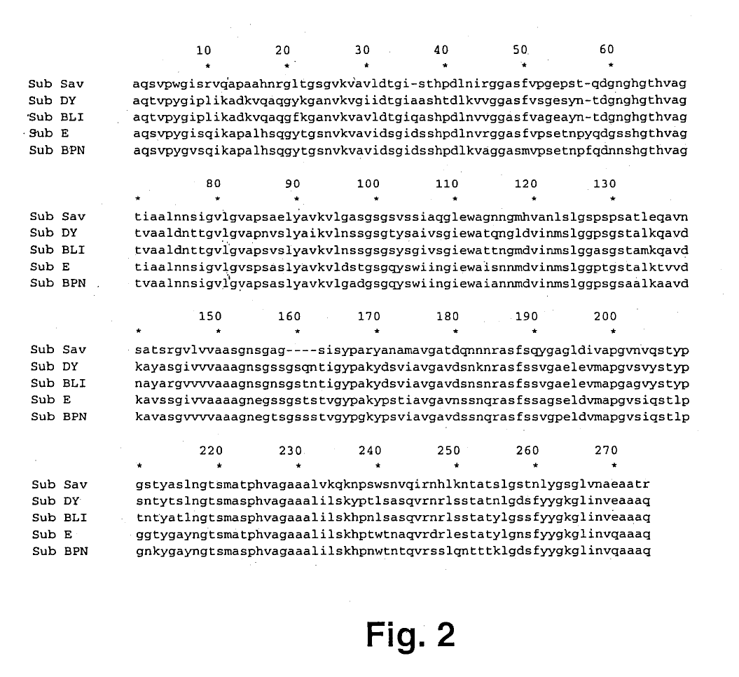

FIG. 3 shows “exemplarily” the improvement of subtilisin variants relatiy to the subtilisin E wild type over 4 rounds of directed evolution towards higher activity in laundry detergents.

FIG. 4 shows the protein degradation performance of subtilisin variant 22 in comparison to wild type subtilisins. All enzymes were applied at concentrations of 2.5 mg/l.

FIG. 5: Determination of the amount of enzyme of variant 50, 1, 4 or 22 compared to the subtilisin E wild type needed in order to achieve a certain protein degradation.

FIG. 6 shows a Michaelis Menten plot of subtilisin variants 1, 4, 22. All enzymes were applied at concentrations of 2.5 mg/l.

FIG. 7 shows a comparison of the protein degradation and solubilization performance between a laundry detergent containing variant 22 and a laundry detergent containing a commercially available subtilisin.

### DETAILED DESCRIPTION OF THE INVENTION

The present invention provides subtilisin variants with improved characteristics compared to those of the respective precursor subtilisin enzymes. These improved properties enable a better utility in cleaning compositions.

In the context of this invention “subtilisins” are serine proteases with low substrate specificity and structural homology to subtilisin E from Bacillus subtilis (Biochim. Biophys. Acta 1543 (2000) 203-222). Serine proteases are characterized by the catalytic triade comprising aspartate-histidine-serine, reading from the amino to the carboxy terminus. Well characterized subtilisins derive from bacterial strains, such as subtilisin E (Bacillus subtilis), subtilisin 309 (Bacillus lentus), subtilisin BPN′ (Bacillus amyloliquefaciens) or subtilisin Carlsberg (Bacillus licheniformis). Derived from different species the amino sequences of naturally occuring subtilisins are not entirely homologous, but differ in amino acid substitutions, insertions and deletions. For the purpose of this invention all positions mentioned in the context of subtilisin are equivalent to the numbering of the mature form of the naturally occuring subtilisin BPN′ of Bacillus amyloliquefaciens.

The amino acids mentioned are abbreviated according to the following list either in one- or in three-letter code.

| | |
|---|---|
| Abbreviations | Amino acid |

| | | |
|---|---|---|
| A | Ala | Alanin |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophane |
| Y | Tyr | Tyrosine |

The present invention provides “variants” of subtilisins. A “variant” in accordance with the invention has an amino acid sequence not found in nature, but is instead derived from the amino acid sequence of a “precursor” enzyme by one or more amino acid substitutions (in the present invention also referred to as “mutation”).

According to the invention, “precursor” subtilisins can be naturally occuring subtilisins such as subtilisin E (Bacillus subtilis strain 168; SEQ ID NO:2), subtilisin BPN′ (Bacillus amyloliquefaciens; SEQ ID NO:1), subtilisin DY (Bacillus subtilis strain DY; SEQ ID NO:3), subtilisin Carlsberg (Bacillus licheniformis; SEQ ID NO:4), subtilisin savinase (Bacillus lentus; SEQ ID NO:5), subtilisin J (Bacillus stearothermophilus; SEQ ID NO:6), alkaline elastase YaB (Bacillus sip. strain YaB; SEQ ID NO:7) and the alkaline protease precursor proteins shown in SEQ ID Nos:8 and 9, or a fragment or derivative thereof, preferably the precursor subtilisin is subtilisin E (SEQ ID NO:2) or a fragment or derivative thereof, or is a recombinant or engineered subtilisin, but also engineered subtilisins having at least 70% homology in its amino acid sequence to a naturally found subtilisin. Furthermore, recombinant subtilisins (being derived from a combination of amino acid sequences of two or more precursor subtilisins) can serve as precursor subtilisins.

By introducing mutations in the amino acid sequence of a precursor subtilisin the characteristics of the resulting subtilisin can be improved. In the context of the present invention, the characteristics of the subtilisin include one, several, or all of the following parameters “Km”, “catalytic activity”, “catalytic efficiency” and “wash performance”. The Km (or “Michaelis Menten constant”) and the catalytic activity are kinetic parameters of an enzyme according to the definitions of Michaelis and Menten (see Fersht, A., Enzyme Structure and Mechanism, W. H. Freeman and Company, New York, 1995). The term Km describes the affinity of the enzymes to its substrate. The “catalytic activity” used herein describes the rate of conversion of the substrate under defined conditions. In the context of this invention, kinetic data are obtained by measuring the hydrolysis of substrates over time. Substrates were fluorescent labeled casein or bovine serum albumin molecules. The degradation was measured over time using confocal fluorescence spectroscopy (see WO9416313). The term “wash performance” used in this invention describes the solubilization and degradation of proteins bound to textiles by the investigated protease applied in a detergent without proteases and compared to the effect of this detergent alone.

In the context of this invention, the term “original amino acid” means the amino acid found in the subtilisin E wild type enzyme at the particular position. It should be noted, that if another subtilisin is to be improved by introducing the amino acid substitutions disclosed in the present invention, the wild type amino acid residue at the particular position might differ from the “original amino acid”. Substitutions are usually described as follows: original amino acid; position; substituted amino acid.

The subtilisin variants generated by means of directed evolution as described in the Experimental Section have surprisingly been found to have improved characteristics as industrial enzymes such as detergent proteases.

The subtilisin variants according to embodiment (1) of the present invention comprise one or a combination of the particular mutations (a) to (e), or mutations at the the respective positions which are homologous to said mutations (a) to (e).

In particular, the mutation

(a) E54D or E54G means that at position 54, glutamic acid is exchanged by an aspartic acid or a glycine;

(b) Q103R or Q103K means that at position 103, glutamine is exchanged by an arginine or a lysine;

(c) T133K means that at position 133, threonine is exchanged by a lysine;

(d) E156K or E156A means that at position 156, glutamic acid is exchanged by a lysine or an alanine; and

(e) Y217H means that at position 217, tyrosine is exchanged by a histidine. “Homologous mutations” according to the present invention are mutations which a person skilled in the art would consider homologous, i.e. equivalent. Thus if the mutated (the newly introduced) amino acid is e.g. a lipophilic amino acid, such as Leu, then other lipophilic amino acids such as Ile are also applicable.

In a preferred embodiment of the invention the subtilisin comprises at least two of the mutations (a) to (e) which includes the following substitution combinations with the numbering of the substitued amio acid resides equivalent to the numbering of Bacillus amyloliquefaciens subtilisin (subtilisin BPN′).

E54D/Q103R, E54G/Q103R, E54D/Q103K, E54G/Q103K, E54D/T133K, E54G/T133K, E54D/E156K, E54G/E156K, E54D/E156A, E54G/E156A, E54D/Y217H, E54G/Y217H, Q103R/T133K, Q103R/E156K, Q103R/E156A, Q103R/Y217H, Q103K/T133K, Q103K/E156K, Q103K/E156A, Q103K/Y217H, T133K/E156K, T133K/E156A, T133K/Y217H, E156K/Y217H, E156A/Y217H, E54D/Q103R/T133K, E54G/Q103R/T133K, E54D/Q103R/E156K, E54G/Q103R/E156K, E54D/Q103R/E156A, E54G/Q103R/E156A, E54D/Q103R/Y217H, E54G/Q103R/Y217H, E54D/Q103K/T133K, E54G/Q103K/T133K, E54D/Q103K/E156K, E54G/Q103K/E156K, E54D/Q103K/E156A, E54G/Q103K/E156A, E54D/Q103K/Y217H, E54G/Q103K/Y217H, E54D/T133K/E156K, E54G/T133K/E156K, E54D/T133K/E156A, E54G/T133K/E156A, E54D/T133K/Y217H, E54G/T133K/Y217H, E54D/E156K/Y217H, E54G/E156K/Y217H, E54D/E156A/Y217H, E54G/E156A/Y217H, Q103R/T133K/E156K, Q103R/T133K/E156A, Q103R/T133K/Y217H, Q103R/E156K/Y217H, Q103R/E156A/Y217H, Q103K/T133K/E156K, Q103K/T133K/E156A, Q103K/T133K/Y217H, Q103K/E156K/Y217H, Q103K/E156A/Y217H, T133K/E156K/Y217H, T133K/E156A/Y217H, E54D/Q103R/T133K/E156K, E54G/Q103R/T133K/E156K, E54D/Q103R/T133K/E156A, E54G/Q103R/T133K/E156A, E54D/Q103R/T133K/Y217H, E54G/Q103R/T133K/Y217H, E54D/Q103R/E156K/Y217H, E54G/Q103R/E156K/Y217H, E54D/Q103R/E156A/Y217H, E54G/Q103R/E156A/Y217H, E54D/Q103K/T133K/E156K, E54G/Q103K/T133K/E156K, E54D/Q103K/T133K/E156A, E54G/Q103K/T133K/E156A, E54D/Q103K/T133K/Y217H, E54G/Q103K/T133K/Y217H, E54D/Q103K/E156K/Y217H, E54G/Q103K/E156K/Y217H, E54D/Q103K/E156A/Y217H, E54G/Q103K/E156A/Y217H, E54D/T133K/E156K/Y217H, E54G/T133K/E156K/Y217H, E54D/T133K/E156A/Y217H, E54G/T133K/E156A/Y217H, Q103R/T133K/E156K/Y217H, Q103R/T133K/E156A/Y217H, Q103K/T133K/E156K/Y217H, Q103K/T133K/E156A/Y217H, E54D/Q103R/T133K/E156K/Y217H, E54D/Q103R/T133K/E156A/Y217H, E54G/Q103R/T133K/E156K/Y217H, E54G/Q103R/T133K/E156A/Y217H, E54D/Q103K/T133K/E156K/Y217H, E54D/Q103K/T133K/E156A/Y217H, E54G/Q103K/T133K/E156K/Y217H, E54G/Q103K/T133K/E156A/Y217H.

More preferably the subtilisin of the invention comprises at least three, even more preferably at least four and most preferably at least five of said mutations (a) to (c).

The subtilisin of the invention with at least two modifications (a) to (e) includes:

(i) a subtilisin according to embodiment (1) which has a mutation (a), preferably the modification E54D, and has at least one (1), preferably at least two (2), more preferably at least three (3) and most preferably at least four (4) additional mutations selected from the mutations (b) to (e) (particularly preferred is a subtilisin according to (i) above which has at least the additional modifications Q103R and E156A);

(ii) a subtilisin according embodiment (1) which has a mutation (b), preferably the modification Q103R, and has at least one (1), preferably at least two (2), more preferably at least three (3) and most preferably at least four (4) additional mutations selected from the mutations (a) and (c) to (e) (particularly preferred is a subtilisin defined above, which has modifications Q103R and E156A and at least one further modification (a), (c) or (e), preferably the additional mutation T133K);

(iii) a subtilisin according to embodiment (1) which has modification (c) and has at least one (1), preferably at least two (2), more preferably at least three (3) and most preferably at least four (4) additional mutations selected from the mutations (a), (b), (d) and (e);

(iv) a subtilisin according to embodiment (1) which has a mutation (d), preferably the modification E156A, and has at least one (1), preferably at least two (2), more preferably at least three (3) and most preferably at least four (4) additional mutations selected from the mutations (a) to (c) and (e); and

(v) a subtilisin according to embodiment (1) which has a mutation (e) and has at least one (1), preferably at least two (2), more preferably at least three (3) and most preferably at least four (4) additional mutations selected from the mutations (a) to (d).

According to the embodiment (2) of the invention, the improved subtilisin variant comprises a combination of at least any two of the following group of amino acid substitutions: E54D, Q103R, T133K, E156A, Y217H.

The subtilisin variants according to embodiments (1) and (2) of the present invention may comprise additional amino acid substitution including substitutions at the following positions: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

Particularly preferred is that the additional mutation is selected from the following group of amino acid substitutions:

V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, I234N, L235I, L235P, T242A, N243S, N243H, N243T, Q245L, D248V, R247H, R249S, S252G, T253S, N259H, Y263C, Y263N, K265N, L267F, N269H, N269I, N269D, Q271H, Q271L, A272P, Q275L.

In particularly preferred embodiment (3) of the invention, the improved subtilisin is one of the following variants comprising particular combinations of amino acid substitutions (with the numbering of the substituted amino acid resides equivalent to the numbering of subtilisin BPN′):

Variant 1:Y6N/I31L/S38T/F50I/Q103R,

variant 2: P14T/Q103R/T130I/Q185H/K265N,

variant 3: V511/Q103R/E156A,

variant 4: Q103R/V138A/A150V/E156A/Y217H/Q271H,

variant 5: S9P/R45S/Q103R/T130A/E156G/S183N/Y217H,

variant 6: G61S/S78P/Q103R/E156A/

variant 7: E54D/I79F/Q103R/E156G/Y217H/N243S,

variant 8: S78P/L90I/Q103R/E156A,

variant 9: Q103K/T130A/E156G/L209H,

variant 10:Q103K/E156A/T242A/N259H,

variant 11: N25D/Q103K/E156A,

variant 12: P14T/A15G/E54D/Q103K/T130A/E156G/S252G/Q271L,

variant 13: Q103K/T133K/N181S/S252G/Q271L,

varinat 14: N43I/G61S/S78P/Q103K/T130A/E156G/S204T

variant 15: N43S/E54D/Q103R/S145G/E156K/G157D/M199V/Y217H/D248V,

variant 16: S18T/N43S/Q103R/V138A/A150V/E156A/Y217H/Q271L,

variant 17: P14T/A15V/H17L/Q103R/T130A/E156A,

variant 18: Q103R/V138A/A150V/E156A/Y217H/Q271H,

variant 19: Q103R/N181S/S204T/Y217H/T224A/A228T,

variant 20: V4A/E54D/G61S/Q103R/E156A/S183N/Y217H,

variant 21: S9P/E54G/Q103R/I111V/E156A/Y217H,

variant 22: E54D/Q103R/T133K/E156A/Y217H,

variant 23: S24T/G61S/Q103R/E156A/S183N/Y217H/T253S,

variant 24: G20D/N43T/E54D/Q103R/T130A/E156A/S183N/Y217H/L235I,

variant 25: V4A/E54D/G61S/Q103R/S145G/E156A/S183N/Y217H,

variant 26: Q103R/V138A/A150V/E156A/Y217H/D248V/R249S,

variant 27: P14T/A15V/I31L/N43S/E54D/Q103R/E156A,

variant 28: Q19H/I31L/E54D/Q59L/S63P/Q103R/Y104N/T130A/E156G/S161N/S183N/N243H/L267F/N269H,

variant 29: E54D/Q103R/I111N/N118Y/T130A/E156A/Q185H/I234N/L235P/Q245L/N259H,

variant 30: E54D/Q103R/I111N/N118Y/T130A/E156A/Q185H,

variant 31: E54D/T130A/V149I/E156A/S183N/D197G/Q206H/N218I/N269I,

variant 32: E54D/Q103R/T133K/K136N/E156A/N269D,

variant 33: Y6N/P14T/A15V/I31L/N43S/E54D/Q103R/E156A,

variant 34: P14T/A15V/E54D/Q103R/E156A/Y167F/Y217H/N218T,

variant 35: P14T/A15V/I31L/E54D/Q103R/N118Y/E156A/S191G/N243T/Y263C/N269H,

variant 36: G7S/S9A/Q103R/T133K/E156A/Y217H/T224A/Y263N/A272P,

variant 37: E54D/Q103R/T133K/K136N/E156A/Y217H,

variant 38: S9P/Q103R/T133K/E156A/T164S/S183N/Y217H,

variant 39: E54D/Q103R/T133K/E156A/N184Y/Q206L/Y217H/K265N,

variant 40: P5L/Y6H/S49G/Q103R/T133K/E156A/Y217H,

variant 41: E54G/Q103R/I11V/V138A/A150V/E156A/Y217H/L235I,

variant 42:S89P/Q103R/E156A/Q271H,

variant 43: I31L/N43S/N56S/Q103R/V138A/A150V/E156A/Y217H/Q271H,

variant 44: P14T/A15V/E54D/Q103R/E156A/Y217H/Q271H,

variant 45: E54D/Q103R/T133K/E156A,

variant 46: E54D/Q103R/T133K,

variant 47: V4A/E54D/G61S/Q103R/S183N,

variant 48: S9P/E54D/Q103R/I111 V/S183N/D248V/R249S,

variant 49: N43S/E54D/Q103R/S145G/E156K/G157D/M199V/Y217H/D248V

variant 50: S145G/E156K/R247H

Among the variants 1 to 50 those derived from subtilisin E as shown in SEQ ID NOs:10 to 59 are particularly preferred.

The substitutions in the amino acid sequence of a precursor subtilisin result in subtilisin variants that surprisingly show enhanced characteristics compared to the precursor subtilisin. The subtilisin variants have distinguishing properties, such as improved catalytic actitvity as compared to the precursor subtilisins. The subtilisin variants of interest in this invention have a lower Km and a higher catalytic activity in comparision to the precursorsubtilisin. Furthermore, the subtilisin variants of interest in this invention surprisingly show an improved wash perfomance in laundry detergent. The improvement of wash performance is connected with a better result in proteinaceous stain removal or otherwise stated less of the subtilisin variant has to be added to obtain the same result as with the precursor subtilisin. Furthermore, these variants show a better stability in presence of typical ingredients of cleaning composition as oxidizing agents.

Moreover, the invention comprises also the use of the subtilisin variants as defined herein before as additive in a cleaning composition. This cleaning composition may be used as laundry detergent or a dishwasher detergent.

The invention will now be described in greater detail in the following examples which are, however, not to be construed to limit the invention.

### EXAMPLES

### Example I

### Cloning of the Subtilisin E Gene and Extracelluar Expression by B. subtilis

The aprE gene coding for subtilisin E was amplified by PCR from the genome of Bacillus subtilis strain 168 (DSM #402). Amplification was performed for 20 cycles using a 5:1 mixture of Taq and Pfu polymerases while B. subtilis cells served as templates. The gene was ligated into the vector pBV1 behind the P43 promotor. The vector pBV1 was constructed as follows: The pMB1 origin from pUC19 (ATCC 37254) was PCR amplified (positions 763-1601) and introduced into the PvuII site of pUB110 (ATCC 37015). The fragment between SapI and BglII was removed from this vector thereby deleting its mob gene (Colin R. Harwood, editor (1989) Bacillus, Plenum Press, New York). Then, an insert containing the P43 promoter from the cdd gene of B. subtilis, the signal sequence and the terminator from the subtilisin E gene of B. subtilis, as well as a short multiple cloning site between the signal sequence and the terminator was introduced into the unique SphI site, resulting in the vector pBV1.

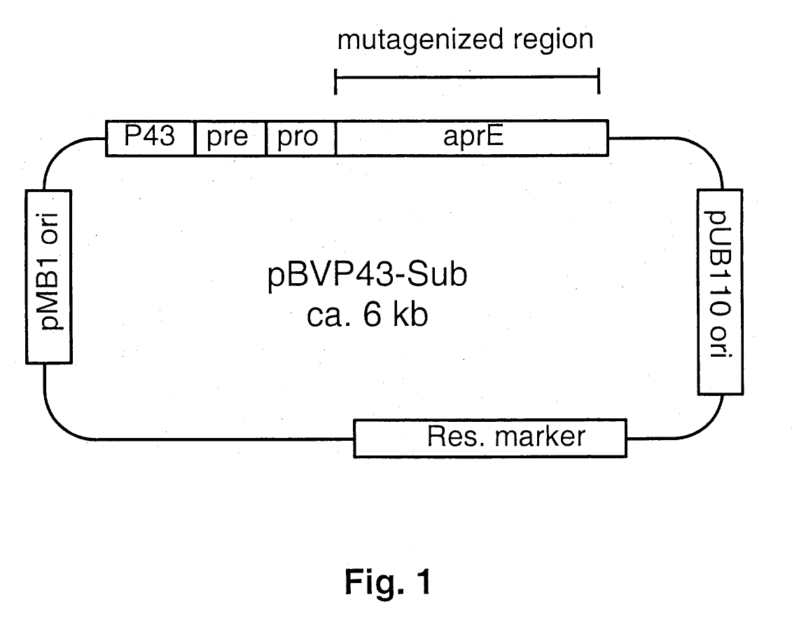

The wild type subtilisin E gene (Seq. ID:60) from the genome of Bacillus subtilis strain 168 (DSM #402) without the signal sequence as well as any other subtilisin variant was introduced in frame with the signal sequence into the multiple cloning site resulting in the vector pBVP43-Sub (J. Biol. Chem. 1984, 259 (13), 8619-8625; Biotechnol. Bioeng. 1999, 62 (1), 87-96). Mutagenesis was restricted to the DNA sequence encoding the mature subtilisin (amino acid sequence of subtilisin E in Seq. ID: 2) without affecting the N-terminal signal sequence. The vector construct is schematically shown in FIG. 1. Identity of the intermediates as well as the final product of the cloning procedure was confirmed by means of DNA cycle sequencing using the didesoxynucleotide method. Functionality of the construct was confirmed by plating transformants of pBVP43-Sub in an aprE deficient B. subtilis strain (Harwood and Cutting (1990) Molecular Biological Methods for Bacillus, J. Wiley and Sons, New York) on LB agar containing 1% skim milk and the appropriate antibiotics. Subtilisin activity resulted in cleared halos around the Bacillus subtilis colonies. Expression of subtilisin E or a subtilisin variant was done by inoculating complex media containing NaCl, trypton and yeast extract with a aprE deficient B.

subtilis clone transformed with a pBVP43-Sub plasmid encoding the particular variant, and incubation in Erlenmeyer flasks for 24 to 48 h at 30° C. with continuous shaking. After separation of the cells by centrifugation, either the supernatant was used directly for enzyme characterization, or the enzyme was further purified by salt precipitation, ion exchange chromatography, dialysis and subsequent lyophilization. Concentration of the enzyme in solution was determined by standard protein quantification methods such as the Bradford test. Purity and integrity of the final enzyme was analyzed by SDS polyacrylamide gel electrophoresis.

### Example II

### Generation of Improved Subtilisin Variants by Means of Directed Evolution

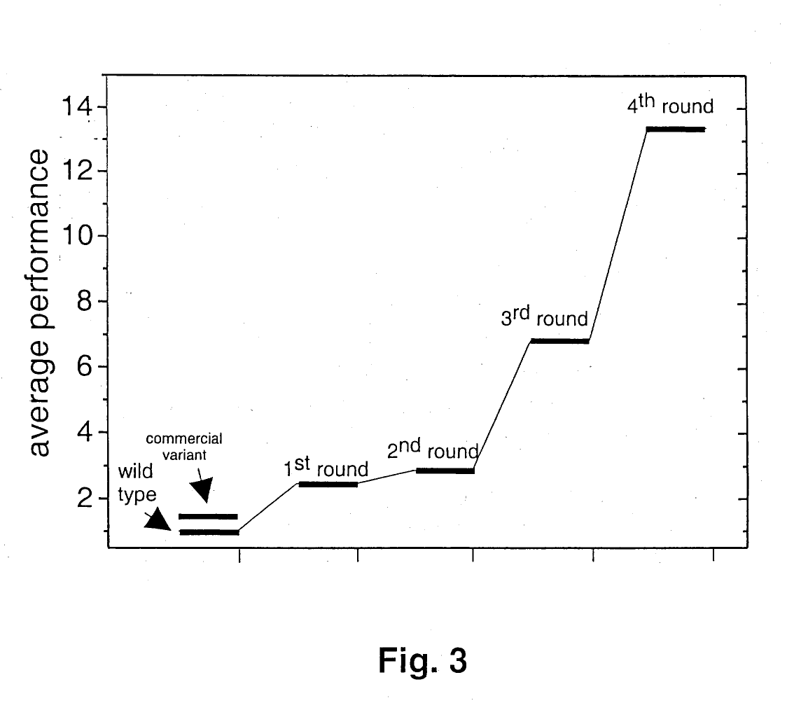

Subtilisin variants with improved characteristics were generated by means of directed evolution essentially following the protocol provided in WO9218645 in combination with screening methods as provided in WO9416313 or WO9934195. According to these methods, the gene coding for subtilisin E was mutagenized by replication using a DNA polymerase with an error rate in the region of the error threshold. Typically, a modified PCR was used for this purpose. Ligation into the vector was done using 300 fmol vector, 1500 fmol insert, 2 μl of 10× Ligation buffer (500 mM Tris-HCl, pH 7.5; 100 mM MgCl₂; 100 mM DTT; 10 mM ATP, 250 μg/ml BSA), 5 Weiss Units of T4 DNA ligase (MBI Fermentas), ad 20 μl aqua dest, by incubation for 2 h at room temperature, followed by heat inactivation for 10 min at 65° C., and ethanol precipitation. The ligation mixture was then transformed into electrocompetent E. coli XL1-Blue. After preparation of the resulting DNA product from E. coli XL1-Blue cells, the library was transformed into an aprE deficient B. subtilis strain. Transformed B. subtilis cells containing the library were then distributed into the compartments of a suitable sample carrier. Subtilisin variants produced by B. subtilis cells in the particular compartments were then examined under conditions relevant to the final application by ultra high throughput screening with confocal fluorescence spectroscopy (see WO9416313 or WO9934195). Genes coding for subtilisin variants with improved characteristics under the screening conditions were subsequently selected and isolated by means of PCR amplification, recloned and re-mutagenized. This cycle of mutagenesis, screening and selection was repeated several times. Optionally, the genes coding for selected variants were analyzed by sequencing. As a further option, mutations occurring in different variants selected in a screening round were combined by means of standard restriction and ligation methods or alternatively by means of homologous recombination methods, e.g. as provided in WO0134835. An example of the continuous improvement of subtilisin characteristics by directed evolution over 4 rounds is shown in FIG. 3.

### Example III

### Protein Degradation Performance of Improved Subtilisin Variants in Detergent

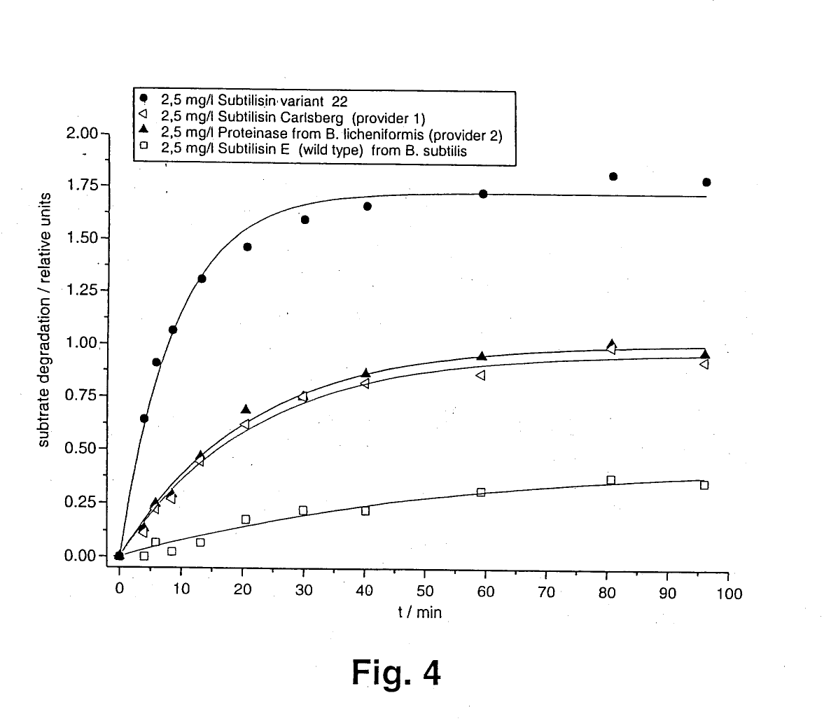

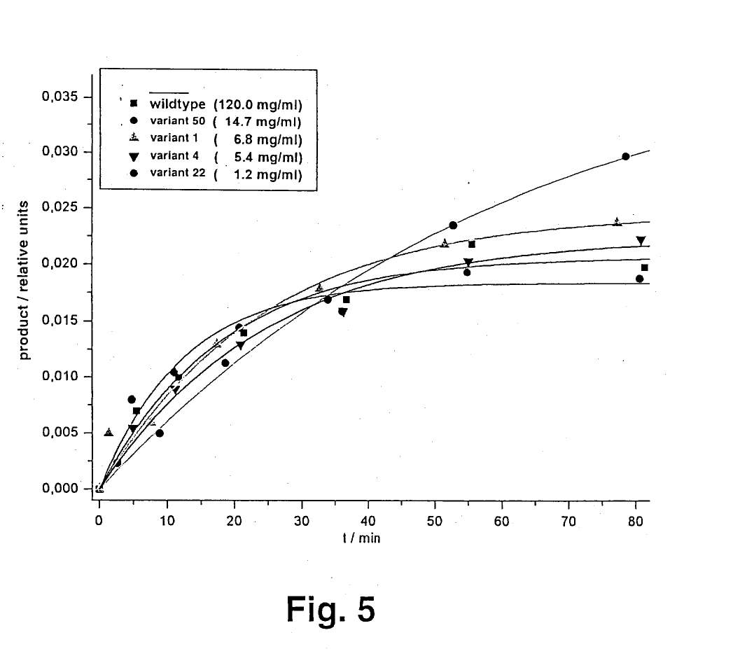

The improvement of subtilisin variants was analyzed by measuring the rate of hydrolysis of protein substrates. Bovine serum albumin (BSA, obtained from Sigma) as an exemplary globular protein was labeled with the fluorescent dye TIRTC (obtained from Sigma) resulting in a labeling degree of approximately one mol dye per mol protein. The degradation of the labelled protein was measured in solution by single-molecule anisotropy, fluorescence correlation spectroscopy and fluorescence intensity measurements using a confocal fluorescence spectroscopy set-up (see WO9416313). For further evaluation, FITC-labelled casein (casein obtained from Sigma, FITC obtained from Fluka) was used as a substrate. For analyzing the characteristics of enzyme variants under washing conditions, tests were carried out in a freshly prepared typical laundry detergent solution. In FIG. 4 the degradation of an improved subtilisin variant is shown in comparison to the wild type as well as another commercially applied subtilisin, Subtilisin Carlsberg from B. licheniformisobtained from two sources, at typical concentrations. In FIG. 5 another set of four variants is compared in terms of the amount of enzyme needed to catalyze protein degradation with the same rate. As can be easily seen, the best variant out of this set has the same catalytic activity at 1/100 concentration compared to the wild type. Table I gives an overview of several variants and their degradation performance is measured as protein degradation in detergent after a periode of 30 minutes normalized to the wild type.

TABLE I

| subtilisin/ | relative protein |
|---|---|
| SEQ ID NO | degradation performance |
| wild type/2 | 1.00 |
| variant 1/10 | 3.91 |
| variant 2/11 | 1.90 |
| variant 3/12 | 4.34 |
| variant 4/13 | 6.85 |
| variant 5/14 | 2.18 |
| variant 7/16 | 5.29 |
| variant 8/17 | 2.26 |
| variant 9/18 | 2.06 |
| variant 10/19 | 4.56 |
| variant 11/20 | 4.26 |
| variant 12/21 | 2.81 |
| variant 13/22 | 1.84 |
| variant 15/24 | 4.30 |
| variant 16/25 | 6.57 |
| variant 17/26 | 5.85 |
| variant 18/27 | 6.60 |
| variant 19/28 | 6.35 |
| variant 20/29 | 7.45 |
| variant 21/30 | 8.50 |
| variant 22/31 | 8.37 |
| variant 23/32 | 8.04 |
| variant 24/33 | 7.63 |
| variant 25/34 | 7.74 |
| variant 26/35 | 7.79 |
| variant 27/36 | 6.16 |
| variant 28/37 | 5.98 |
| variant 29/38 | 5.75 |
| variant 30/39 | 6.73 |
| variant 31/40 | 5.95 |
| variant 32/41 | 6.77 |
| variant 33/42 | 6.67 |
| variant 34/43 | 7.24 |
| variant 35/44 | 4.12 |
| variant 36/45 | 6.89 |
| variant 37/46 | 7.45 |
| variant 38/47 | 6.13 |
| variant 39/48 | 7.22 |
| variant 40/49 | 5.91 |
| variant 41/50 | 5.73 |
| variant 42/51 | 3.77 |
| variant 43/52 | 4.99 |
| variant 44/53 | 3.36 |

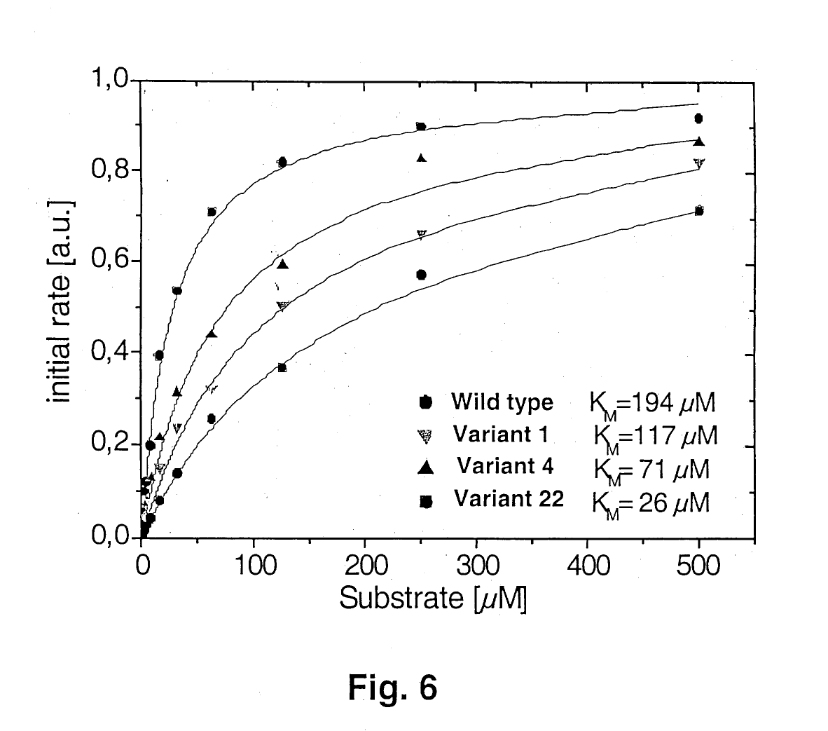

FIG. 6 and Table II compare the Km of some of these variants with the Km of the wild type enzyme.

TABLE II

| Subtilisin/SEQ ID NO | Km [μM] |
|---|---|
| Subtilisin E (wild type)/2 | 194 |
| Variant 1/10 | 117 |
| Variant 4/13 | 71 |
| Variant 22/31 | 26 |

### Example IV

### Effect of Improved Variants on Protein Solubilization and Subsequent Degradation from Textiles

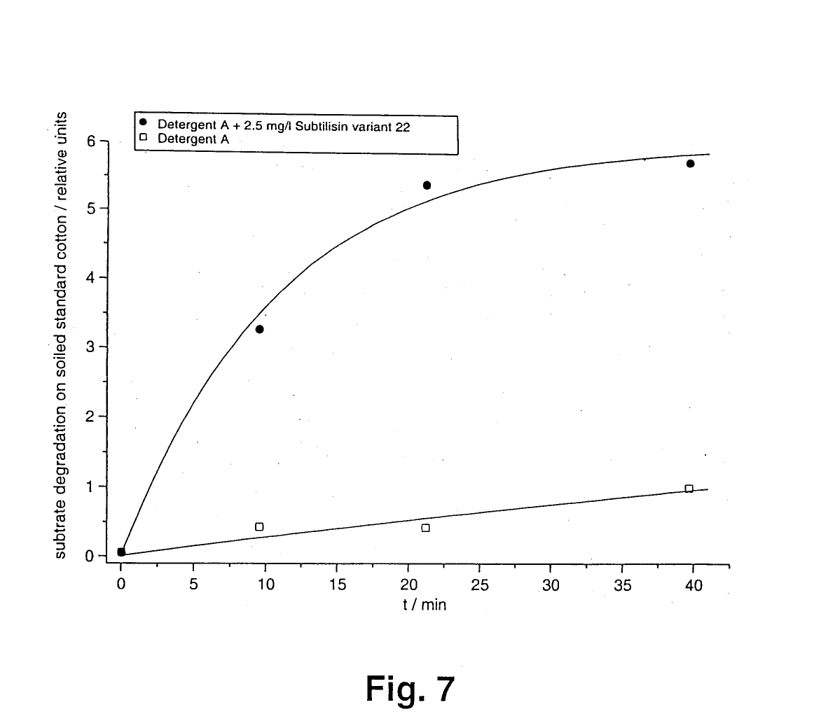

In order to analyze the degradation of proteins bound to textile surfaces and, thereby, the efficieny of a particular subtilisin variant in removing protein stains from soiled textiles, a solid phase assay was used. Standard cotton (Nr. 10A, wfk Testgewebe GmbH, Christenfeld 10, D41379 Brüggen-Bracht, Germany) was impregnated with 20% hen egg solution containing TIRTC labelled BSA. Then the cotton was cut into pieces, dried, washed three times with laundry detergent containing no protease, dried again and then incubated with laundry detergent containing the particular subtilisin variant, or commercially available subtilisin variant, both at 2.5 mg/l, or no protease as control. The release of proteins from the textile and the further degradation in solution was then measured by single-molecule anisotropy, fluorescence correlation spectroscopy and fluorescence intensity measurements using a confocal fluorescence spectroscopy set-up (see WO9416313). FIG. 7 compares the rate of release of labelled protein fragments catalyzed by a particular variant with the rate of a comercially available protease.

1. A modified subtilisin which has at least one mutation in an amino acid sequence of a precursor subtilisin at a position numbered according to the amino acid sequence of the mature subtilisin BPN′ shown in SEQ ID NO:1, wherein said at least one mutation is selected from (a) E54D or E54G, (b) Q103R or Q103K, (c) T133K, (d) E156K or E156A, and (e) Y217H, or mutations at the respective positions which are homologous to said mutations (a) to (e), provided that in case of mutations E54D, Q103R, E156K or E156A the modified subtilisin has at least one more of said mutations (a) to (e).

2. The modified subtilisin of claim 1, which has at least two (2) of said mutations (a) to (e).

3. The modified subtilisn of claim 1, which has at least three (3) of said mutations (a) to (e).

4. The modified subtilisins of claim 1, which has at least four (4) of said mutations (a) to (e).

5. The modified subtilisins of claim 1, which has at least five (5) of said mutations (a) to (e).

6. The modified subtilisin of claim 1, which has at least two (2) of the following group of amino acid substitutions: E54D, Q103R, T133K, E156A, Y217H.

7. The modified subitilisn of claim 1, which has at least three (3) of the following group of amino acid substitutions: E54D, Q103R, T133K, E156A, Y217H.

8. The modified subtilisins of claim 1, which has at least four (4) of the following group of amino acid substitutions: E54D, Q103R, T133K, E156A, Y217H.

9. The modified subtilisins of claim 1, which has at least five (5) of the following group of amino acid substitutions: E54D, Q103R, T133K, E156A, Y217H.

10. The modified subtilisin of any one of claim 1, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

11. The modified subtilisin of claim 1, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, I234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

12. The modified subtilisin according to claim 1, which has a mutation (a) and has at least one (1) additional mutations selected from the mutations (b) to (e).

13. The modified subtilisins according to claim 12, which has at least two (2) additional mutations selected from the mutations (b) to (e).

14. The modified subtilisins according to claim 12, which has at least three (3) additional mutations selected from the mutations (b) to (e).

15. The modified subtilisins according to claim 12, which has at least four (4) additional mutations selected from the mutations (b) to (e).

16. The modified subtilisin according to claim 12, which has at least the additional mutations Q103R and E156A.

17. The modified subitlisin of claim 12, which has the modification E54D.

18. The modified subtilisin according to claim 14, which has at least the additional mutations Q103R and E156A.

19. The modified subtilisin of any one of claim 12, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

20. The modified subtilisin of claim 12, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, I234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

21. The modified subtilisin according to claim 1, which has a mutation (b) and has at least one (1) additional mutation selected from the mutations (a) and (c) to (e).

22. The modified subtilisins according to claim 21, which has at least two (2) additional mutations selected from the mutations (a) and (c) to (e).

23. The modified subtilisins according to claim 21, which has at least three (3) additional mutations selected from the mutations (a) and (c) to (e).

24. The modified subtilisins according to claim 21, which has at least four (4) additional mutations selected from the mutations (a) and (c) to (e).

25. The modified subtilisins according to claim 21, which has the modification Q103R.

26. The modified subtilisin according to claim 21, which has modifications Q103R and E156A and at least one further modification (a), (c) or (e).

27. The modified subtilisins according to claim 26, which has the further mutation T133K.

28. The modified subtilisin of any one of claim 21, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

29. The modified subtilisin of claim 21, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, 1234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

30. The modified subtilisin according to claim 1, which has modification (c) and has at least one (1) additional mutation selected from the mutations (a), (b), (d) and (e).

31. The modified subtilisin according to claim 1, which has modification (c) and has at least two (2) additional mutations selected from the mutations (a), (b), (d) and (e).

32. The modified subtilisin according to claim 1, which has modification (c) and has at least three (3) additional mutations selected from the mutations (a), (b), (d) and (e).

33. The modified subtilisin according to claim 1, which has modification (c) and has at least four (4) additional mutations selected from the mutations (a), (b), (d) and (e).

34. The modified subtilisin of any one of claim 30, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

35. The modified subtilisin of claim 30, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, I234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

36. The modified subtilisin according to claim 1, which has a mutation (d) and has at least one (1) additional mutation selected from the mutations (a) to (c) and (e).

37. The modified subtilisin according to claim 36, which has a mutation (d) and has at least two (2) additional mutations selected from the mutations (a) to (c) and (e).

38. The modified subtilisin according to claim 36, which has a mutation (d) and has at least three (3) additional mutations selected from the mutations (a) to (c) and (e).

39. The modified subtilisin according to claim 36, which has a mutation (d) and has at least four (4) additional mutations selected from the mutations (a) to (c) and (e).

40. The modified subtilisins according to claim 36, which has the modification E156A.

41. The modified subtilisin of any one of claim 36, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

42. The modified subtilisin of claim 37, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, 1234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

43. The modified subtilisin according to claim 1, which has a mutation (e) and has at least one (1) additional mutation selected from the mutations (a) to (d).

44. The modified subtilisin according to claim 43, which has a mutation (e) and has at least two (2) additional mutations selected from the mutations (a) to (d).

45. The modified subtilisin according to claim 43, which has a mutation (e) and has at least three (3) additional mutations selected from the mutations (a) to (d).

46. The modified subtilisin according to claim 43, which has a mutation (e) and has at least four (4) additional mutations selected from the mutations (a) to (d).

47. The modified subtilisin of claim 43, which further has mutation at the following amino acid residue positions of the precursor subtilisin: 4, 5, 6, 7, 9, 14, 15, 17, 18, 19, 20, 24, 25, 31, 38, 43, 45, 49, 50, 51, 54 modified differently as compared to mutation (a), 56, 59, 61, 63, 78, 79, 89, 90, 101, 103 modified differently as compared to mutation (b), 104, 111, 118, 130, 133 modified differently as compared to mutation (c), 136, 138, 145, 149, 150, 156 modified differently as compared to mutation (d), 157, 161, 164, 167, 173, 181, 183, 184, 185, 191, 197, 199, 204, 206, 209, 217 modified differently as compared to mutation (e), 218, 224, 228, 234, 235, 242, 243, 245, 247, 248, 249, 251, 252, 253, 259, 263, 265, 271, 267, 269, 271, 272, 273 and 275.

48. The modified subtilisin of claim 43, which further has one or more of the following mutations: V4A, P5L, Y6N, Y6H, G7S, S9P, S9A, P14T, A15G, A15V, H17L, S18T, Q19H, G20D, S24T, N25D, I31L, S38T, N43S, N43I, N43T, R45S, S49G, F50I, V51I, N56S, Q59L, G61S, S63P, S78P, I79F, S89P, L90I, Y104N, I111V, I111N, N118Y, T130I, T130A, K136N, V138A, S145G, V149I, A150V, E156G, G157D, S161N, T164S, Y167F, N181S, S183N, N184Y, Q185H, S191G, D197G, M199V, S204T, Q206H, Q206L, L209H, N218I, N218T, T224A, A228T, 1234N, L235I, L235P, T242A, N243S, N243H, N243T, Q245L, R247H, D248V, R249S, S252G, T253S, N259H, Y263C, Y263N, K265N, L267F, N269H, N269I, N269D, Q271H, Q271L, A272P and Q275L.

49. The modified subtilisin according to claim 1, which has one of the following combinations of mutations:
V51I/Q103R/E156A, Q103R/V138A/A150V/E156A/Y217H/Q271H, S9P/R45S/Q103R/T130A/E156G/S183N/Y217H, G61S/S78P/Q103R/E156A/E54D/I79F/Q103R/E156G/Y217H/N243S, S78P/L90I/Q103R/E156A, Q103K/T130A/E156G/L209H, Q103K/E156A/T242A/N259H, N25D/Q103K/E156A, P14T/A15G/E54D/Q103K/T130A/E156G/S252G/Q271L, Q103K/T133K/N181S/S252G/Q271L, N43I/G61S/S78P/Q103K/T130A/E156G/S204T N43S/E54D/Q103R/S145G/E156K/G157D/M 199V/Y217H/D248V, S18T/N43S/Q103R/V138A/A150V/E156A/Y217H/Q271L, P14T/A15V/H 17L/Q103R/T130A/E156A, Q103R/V138A/A150V/E156A/Y217H, Q103R/N181S/S204T/Y217H/T224A/A228T, V4A/E54D/G61S/Q103R/E156A/S183N/Y217H, S9P/E54G/Q103R/I111V/E156A/Y217H, E54D/Q103R/T133K/E156A/Y217H, S24T/G61S/Q103R/E156A/S183N/Y217H/T253S, G20D/N43T/E54D/Q103R/T130A/E156A/S183N/Y217H/L235I, V4A/E54D/G61S/Q103R/S145G/E156A/S183N/Y217H, Q103R/V138A/A150V/E156A/Y217H/D248V/R249S, P14T/A15V/I31L/N43S/E54D/Q103R/E156A, Q19H/I31L/E54D/Q59L/S63P/Q103R/Y104N/T130A/E156G/S161N/S183N/-N243H/L267F/N269H, E54D/Q103R/I111N/N118Y/T130A/E156A/Q185H/I234N/L235P/Q245L/N259H, E54D/Q103R/I111N/N118Y/T130A/E156A/Q185H, E54D/T130A/V149I/E156A/S183N/D197G/Q206H/N218I/N269I, E54D/Q103R/T133K/K136N/E156A/N269D, Y6N/P14T/A15V/I31L/N43S/E54D/Q103R/E156A, P14T/A15V/E54D/Q103R/E156A/Y167F/Y217H/N218T, P14T/A15V/I31L/E54D/Q103R/N118Y/E156A/S191G/N243T/Y263C/N269H, G7S/S9A/Q103R/T133K/E156A/Y217H/T224A/Y263N/A272P, E54D/Q103R/T133K/K136N/E156A/Y217H, S9P/Q103R/T133K/E156A/T164S/S183N/Y217H, E54D/Q103R/T133K/E156A/N 184Y/Q206L/Y217H/K265N, P5L/Y6H/S49G/Q103R/T133K/E156A/Y217H, E54G/Q103R/I111V/V138A/A150V/E156A/Y217H/L235I, S89P/Q103R/E156A/Q271H, 131L/N43S/N56S/Q103R/V138A/A150V/E156A/Y217H/Q271H, P14T/A15V/E54D/Q103R/E156A/Y217H/Q271H, E54D/Q103R/T133K/E156A, E54D/Q103R/T133K, V4A/E54D/G61S/Q103R/S183N, S9P/E54D/Q103R/I111V/S13N/D248V/R249S and N43S/E54D/Q103R/S145G/E156K/G157D/M199V/Y217H/D248V.

50. A modified subtilisin comprising the following combination of mutations in an amino acid sequence of a precursor subtilisin at a position numbered according to the amino acid sequence of the mature subtilisin BPN′ shown in SEQ ID NO:1:
Y6N/I31L/S38T/F50I/Q103R, P14T/Q103R/T130I/Q185H/K265N, and S145G/E156K/R247H.

51. The modified subtilisin according to claim 1 or 50, wherein the precursor subtilisin is subtilisin E (Bacillus subtilis strain 168; SEQ ID NO:2), subtilisin BPN′ (Bacillus amyloliquefaciens; SEQ ID NO:1), subtilisin DY (Bacillus subtilis strain DY; SEQ ID NO:3), subtilisin Carlsberg (Bacillus licheniformis; SEQ ID NO:4), subtilisin savinase (Bacillus lentus; SEQ ID NO:5), or a fragment or derivative thereof, or is a recombinant or engineered subtilisin.

52. The modified subtilisins according to claim 51, wherein the precursor subtilisin is subtilisin E (SEQ ID NO:2) or a fragment or derivative thereof.

53. The modified subtilisin according to claim 52 which has the sequence shown in SEQ ID NOs:10 to 59.

54. A DNA coding for the modified subtilisin of claim 1.

55. A vector comprising the DNA of claim 54.

56. A microorganism which comprises the DNA of claim 54.

57. A microorganism which comprises the vector of claim 55.

58. A method for producing a modified subtilisin, which comprises culturing the microorganism of claim 56.

59. A method for producing a modified subtilisin, which comprises culturing the microorganism of claim 57.

60. A detergent composition comprising a modified subtilisin of claim 1.

61. A method for degrading proteinaceous material, which comprises treating the proteinaceous material with a modified subtilisin of claim 1.
